(19) United States
(12) Patent Application Publication
(10) Pub. No.: US 2025/0114549 A1
(43) Pub. Date: Apr. 10, 2025

(54) Emergency-Use Respiratory Device

(71) Applicant: Baylor University, Waco, TX (US)

(72) Inventors: Matthew R. Brantley, China Spring, TX (US); Raymond Curtice, Woodway, TX (US)

(73) Assignee: Baylor University, Waco, TX (US)

(21) Appl. No.: 18/984,380

(22) Filed: Dec. 17, 2024

Related U.S. Application Data
(63) Continuation-in-part of application No. 18/143,352, filed on May 4, 2023, now Pat. No. 12,171,939.
(60) Provisional application No. 63/338,964, filed on May 6, 2022.

(51) Int. Cl. A61M 16/00

(52) U.S. Cl. CPC A61M 16/024; A61M 16/0003

(57) ABSTRACT

A portable ventilator device suitable for emergency use, such as cardiac pulmonary resuscitation and well as ventilation in non-cardiac induced medical events. The device is connected to a display screen that receives simple inputs from an operator, including height, weight, or sex of the distressed patient, but does not require input of more complicated variables, such as tidal volume, respiratory rate, inspiratory airflow, or positive end-expiratory pressure. Based on the input height, weight, and/or sex, the device automatically correlates the input values with probably values for tidal volume, respiratory rate, inspiratory airflow, or positive end-expiratory pressure, based previous data point correlations. The device then begins delivering air from a ventilator to the patient based on the estimated, correlated values. While the device utilizes simple inputs, it still capable of operating in multiple modes with both pressure control and volume control.

### CROSS REFERENCE TO RELATED APPLICATIONS

This application is related to and claims priority from the following U.S. patent application. This application is a continuation-in-part of U.S. patent application Ser. No. 18/143,352, filed May 4, 2023, which claims priority to and the benefit of U.S. Provisional Patent Application No. 63/338,964, filed May 6, 2022, each of which is incorporated herein by reference in its entirety.

### BACKGROUND OF THE INVENTION

### 1. Field of the Invention

The present invention relates to respiratory devices and methods, and more specifically to portable ventilators having simplified inputs and therefore being capable of being operated by non-medical professionals without prior training.

### 2. Description of the Related Art

It is generally known in the prior art to provide ventilators for assisting breathing by providing air into a patient's lungs. Both positive pressure ventilators, which push air directly into the lungs, and negative pressure ventilators, which help to allow air to passively be drawn into the lungs, are known in the art. Some positive pressure ventilators only focus on pushing air into the lungs in sync with a patient's breathing, while others, especially invasive mechanical ventilators, take over the breathing process more thoroughly, pushing in air and pulling out carbon dioxide breathed out by the patient.

U.S. Pat. No. 10,245,437 for System and method for providing noninvasive ventilation by inventors Kantor et al., filed Nov. 10, 2013 and issued Oct. 22, 2015, discloses a system that includes both an Airway and Ventilation device (AV) and an Automated External Defibrillator (AED) device. The system allows minimally trained persons to operate it in emergency situations involving respiratory failure and/or cardiac arrhythmias. An integral part of the AV of the system is a face mask manufactured in two parts: a face attachment unit configured to attach to the patient's face and a mask body that is releasably connected to the face attachment unit by a quick release mechanism allowing quick removal of the mask body from the face attachment unit, leaving only the face attachment unit attached to the patients' face, in order to address urgencies such as vomiting. After vomiting ceases and is cleared, then the mask body may be reattached to continue ventilation.

U.S. Pat. No. 7,980,244 for Emergency pulmonary resuscitation device by inventors Boone et al., filed Jul. 17, 2007 and issued Jul. 19, 2011, discloses an emergency pulmonary resuscitation device. The device of the present invention provides emergency breathing for use in techniques such as CPR, and is preferably capable of interaction with an automatic external defibrillator. The device is easy to operate and provides feedback so that it may be used by persons without medical training. The device also works through a simple action, making it possible for it to be inexpensively manufactured and widely disseminated.

U.S. Pat. No. 11,596,753 for Automatic patient ventilator system and method by inventors Sherman et al., filed Nov. 5, 2015 and issued Mar. 7, 2023, discloses ventilator enabling an operator to enter into the microprocessor estimate of a patient's individual characteristic, such as weight, which the microprocessor uses to control delivered tidal volume and other parameters to match the patient. The operator can select one of several ventilator operational modes (intube, mask, CPR). Sensors input data to the microprocessor to maintain parameter optimizations and accuracy. Visual/audible alarms and tools activate when one or more parameters exceed or fail to exceed predetermined values for patient's weight. Manual over-ride is available. The ventilator has a quick start capability in which the operator turns on power, selects the automatic operating mode, enters patient's characteristic, selects control option starting automatic ventilation of proper volumes inhalation/exhalation periods, pressure, and oxy-air mixture.

U.S. Pat. No. 5,915,380 for System and method for controlling the start up of a patient ventilator by inventors Wallace et al., filed Mar. 14, 1997 and issued Jun. 29, 1999, discloses a ventilation control system for controlling the ventilation of a patient. The ventilation control system utilizes a user-friendly user interface for the display of patient data and ventilator status, as well as for entering values for ventilation settings to be used to control the ventilator. Values for ventilation settings entered during set up of the ventilator result in the display of only those further ventilation settings that are appropriate in accordance with the earlier entered settings.

U.S. Patent Pub. No. 2021/0393902 for One-touch ventilation mode by inventors Dong et al., filed Jun. 22, 2021 and published Dec. 23, 2021, discloses systems and methods for one-touch ventilation mode. In examples, settings for a medical ventilator are determined and delivered to a patient with a minimum of one input parameter. The one-touch ventilation mode may reference or apply one or more respiratory mechanics planes to determine desired ventilation parameters. In an example, the input parameter may be mapped to initial ventilation settings on a respiratory mechanics plane. During ventilation delivered according to the initial ventilation settings, ventilation data may be obtained. Based on the ventilation data, one or more ventilation strategies may be implemented, including breath type strategy, alarming strategy, triggering/cycling strategy, and PEEP strategy. Updated ventilation settings may be determined based on the ventilation data and/or the ventilation strategy.

U.S. Pat. No. 8,640,700 for Method for selecting target settings in a medical device by inventor Baker, filed Mar. 23, 2009 and issued Feb. 4, 2014, discloses providing a method for controlling the delivery of a breathing gas to a patient. The method may include regulating the delivery of the breathing gas delivered to the patient, determining a value for a first ventilation parameter, comparing the determined value of the first ventilation parameter to a pre-determined target value for the first ventilation parameter, automatically adjusting the breathing gas delivered to the patient in response to the comparison between the determined value of the first ventilation parameter and the pre-determined target value for the first ventilation parameter, and automatically determining a new target value for the first ventilation parameter based at least in part on the determined value of the first ventilation parameter.

U.S. Patent Pub. No. 2022/0040428 for Ventilation Devices and Systems and Methods of Using Same by inventors Fogarty et al., filed Aug. 18, 2021 and published Feb. 10, 2022, discloses a ventilation system having a mask, a blowing assembly, and a processor. The mask has a mask body and a pressure sensor operatively associated with the mask body and configured to measure pressure within the mask. The mask body defines an inlet opening and a plurality of leak openings. The blowing assembly is positioned in fluid communication with the inlet opening of the mask body and configured to direct air to the inlet opening of the mask body. The processor is positioned in operative communication with the blowing assembly and the pressure sensor of the mask. The processor is configured to selectively control the blowing assembly based upon at least the measured pressure within the mask.

U.S. Pat. No. 10,980,417 for Acute care eco system integrating customized devices of personalized care with networked population based management by inventor Shen, filed May 12, 2015 and issued Apr. 20, 2021, discloses a personalized acute care treatment kit that includes components necessary for a lay caregiver to treat an acute cardiac event. The kit includes a medication box provided with medications selected according to the needs of the owner, a CPR device, a pacemaker, a defibrillator, monitoring and diagnostic devices and a computing device. The computing device is provided with a mobile application that captures patient data from the devices in the kit and automatically sends an alarm to a treatment professional when the patient data exceeds a predetermined threshold and establishes a communication link to with the treatment professional to allow the treatment professional to instruct the lay caregiver in using the contents of the kit to provide acute care.

### SUMMARY OF THE INVENTION

The present invention relates to respiratory devices and methods, and more specifically to portable ventilators having simplified inputs and therefore being capable of being operated by non-medical professionals without prior training.

It is an object of this invention to provide an easy-to-use display interface for a portable ventilator accepting simplified input parameters for a patient that are able to be understood by non-medical users, such as height, weight, and sex, so as to provide temporary, emergency ventilation until medical professionals are able to arrive.

In one embodiment, the present invention is directed to a portable emergency ventilator system, including an inspiratory tubing and an expiratory tubing configured to connect to a patient, an inhalation flow generator connected to the inspiratory tubing, a plurality of sensors connected with the inspiratory tubing, the expiratory tubing, and/or the inhalation flow generator, a microcontroller including a plurality of independent cores, and a microcomputer in communication with the microcontroller, wherein the microcontroller sends sensor data generated by the plurality of sensors to the microcomputer, wherein an artificial intelligence (AI) module of the microcomputer generates a digital twin of the patient with waveforms constructed from the sensor data, and wherein the AI module generates updates to models for operating the ventilator system and wherein the microcomputer sends messages including the updates to the models to the microcontroller.

In another embodiment, the present invention is directed to a portable emergency ventilator system, including a plurality of sensors configured to generate sensor data regarding pressure, flow rate, tidal volume, oxygen content, and/or other information regarding the ventilator system, a microcontroller including a plurality of independent cores, and a microcomputer in communication with the microcontroller, wherein one of the plurality of independent cores of the microcontroller includes a state machine configured to control operating parameters of the ventilator system, wherein the microcontroller sends sensor data generated by the plurality of sensors to the microcomputer, wherein an artificial intelligence (AI) module of the microcomputer generates a digital twin of the patient with waveforms constructed from the sensor data, wherein the AI module generates updates to models for operating the ventilator system and wherein the microcomputer sends messages including the updates to the models to the microcontroller, and wherein the state machine of the microcontroller automatically updates the parameters of the ventilator system based on the messages from the microcomputer.

In yet another embodiment, the present invention is directed to a method for operating a ventilator system, including connecting an inspiratory tubing and an expiratory tubing to a patient, operating an inhalation flow generator to provide air to the patient through the inspiratory tubing, a microcontroller of the ventilator system sending sensor data from a plurality of sensors connected to the inspiratory tubing, the expiratory tubing, and/or the inhalation flow generator to a microcomputer, an artificial intelligence (AI) module of the microcomputer generating a digital twin of the patient with waveforms constructed from the sensor data, and the AI module generating updates to models for operating the ventilator system and sending the updates to the microcontroller.

### BRIEF DESCRIPTION OF THE DRAWINGS

FIG. 1 is a perspective view of a portable ventilator according to one embodiment of the present invention.

FIG. 2 is a schematic diagram of mechanical components of a portable ventilator according to one embodiment of the present invention.

FIG. 3 is a perspective view of a display and input interface of a portable ventilator showing a start-up screen according to one embodiment of the present invention.

FIG. 4 is a perspective view of a display and input interface of a portable ventilator showing a height input interface according to one embodiment of the present invention.

FIG. 5 is a perspective view of a display and input interface of a portable ventilator showing a sex input interface according to one embodiment of the present invention.

FIG. 6 is a perspective view of a display and input interface of a portable ventilator showing a weight input interface according to one embodiment of the present invention.

FIG. 7 is a perspective view of a display and input interface of a portable ventilator showing an instructions screen according to one embodiment of the present invention.

FIG. 8 is a perspective view of a display and input interface of a portable ventilator showing a patient conditions input interface according to one embodiment of the present invention.

FIG. 9 is a perspective view of a display and input interface of a portable ventilator showing an activation screen according to one embodiment of the present invention.

FIG. 10 is a schematic diagram of inspiratory and exhalatory circuits for a ventilator according to one embodiment of the present invention.

FIG. 11 is a schematic diagram of inspiratory and exhalatory circuits for a ventilator according to another embodiment of the present invention.

FIG. 12 is a chart illustrating functions of different parts of a ventilation system across different phases of operation and controlling variables for each phase according to one embodiment of the present invention.

FIG. 13 is a chart illustrating ventilator state machine logic according to one embodiment of the present invention.

FIG. 14 is a schematic diagram illustrating communication between a micro controller and a micro computer for a ventilator according to one embodiment of the present invention.

FIG. 15 is a schematic diagram showing different functions of different logical layers of a ventilation system according to one embodiment of the present invention.

FIG. 16 is a schematic diagram of a system of the present invention.

### DETAILED DESCRIPTION

The present invention relates to respiratory devices and methods, and more specifically to portable ventilators having simplified inputs and therefore being capable of being operated by non-medical professionals without prior training.

In one embodiment, the present invention is directed to a portable emergency ventilator system, including an inspiratory tubing and an expiratory tubing configured to connect to a patient, an inhalation flow generator connected to the inspiratory tubing, a plurality of sensors connected with the inspiratory tubing, the expiratory tubing, and/or the inhalation flow generator, a microcontroller including a plurality of independent cores, and a microcomputer in communication with the microcontroller, wherein the microcontroller sends sensor data generated by the plurality of sensors to the microcomputer, wherein an artificial intelligence (AI) module of the microcomputer generates a digital twin of the patient with waveforms constructed from the sensor data, and wherein the AI module generates updates to models for operating the ventilator system and wherein the microcomputer sends messages including the updates to the models to the microcontroller.

In another embodiment, the present invention is directed to a portable emergency ventilator system, including a plurality of sensors configured to generate sensor data regarding pressure, flow rate, tidal volume, oxygen content, and/or other information regarding the ventilator system, a microcontroller including a plurality of independent cores, and a microcomputer in communication with the microcontroller, wherein one of the plurality of independent cores of the microcontroller includes a state machine configured to control operating parameters of the ventilator system, wherein the microcontroller sends sensor data generated by the plurality of sensors to the microcomputer, wherein an artificial intelligence (AI) module of the microcomputer generates a digital twin of the patient with waveforms constructed from the sensor data, wherein the AI module generates updates to models for operating the ventilator system and wherein the microcomputer sends messages including the updates to the models to the microcontroller, and wherein the state machine of the microcontroller automatically updates the parameters of the ventilator system based on the messages from the microcomputer.

In yet another embodiment, the present invention is directed to a method for operating a ventilator system, including connecting an inspiratory tubing and an expiratory tubing to a patient, operating an inhalation flow generator to provide air to the patient through the inspiratory tubing, a microcontroller of the ventilator system sending sensor data from a plurality of sensors connected to the inspiratory tubing, the expiratory tubing, and/or the inhalation flow generator to a microcomputer, an artificial intelligence (AI) module of the microcomputer generating a digital twin of the patient with waveforms constructed from the sensor data, and the AI module generating updates to models for operating the ventilator system and sending the updates to the microcontroller.

The pandemic of SARS-COV-2, commonly known as COVID-19, taught the public the necessity of respiratory devices, such as ventilators. During the COVID-19 pandemic, hospitals and medical staff frequently needed more ventilators than were available. The needs are even more acute outside the medical centers, such as hospitals and urgent care facilities, where untrained users are called upon to render aid, particularly in emergencies. The last few years found untold millions of people needing respiratory aid outside of the medical centers at least temporarily until medical help is able to be present. The expense of a ventilator discouraged businesses from having such a device on premises and often precluded a homeowner from purchasing one. Further, the complexity of skillfully operating such a sophisticated ventilator to provide sufficient air, yet correctly to avoid harming the patient dissuades many from operating such devices.

Further, a patient's emergency need for ventilation is often a highly stressful event with family members, friends, and coworkers. Logic and deliberation are often compromised. Attempting to operate a sophisticated ventilator that is able to provide the needed air sometimes causes harm to the patient. While some simple devices are commercially sold for ventilation, such known systems lack the ability to handle the varying needs of patient to adequately provide the air without harming the patient that needs the air that the more sophisticated devices in medical centers operated by trained medical personnel are able to provide.

Different ventilators operate in vastly different ways, both in terms of which mechanical circuit elements are included and what operating modes are used to run them. With regard to mechanical configuration, ventilators, particularly those with a dual limb configuration, frequently include a Y-piece connected to both an inhalation tube and an exhalation tube. The inhalation tube is sometimes attachable to a temperature sensor for detecting the incoming air temperature, such that a feedback loop is able to be formed. Ventilators commonly include a heat and moisture exchanger (HME) for humidifying incoming air and, occasionally, the temperature sensor is placed where the air exits the humidifier. Some ventilation circuits also include heated wires running the length of the tubing in order to maintain a consistent temperature. The inhalation tube or the Y-piece also sometimes includes a port for administering nebulized drugs. Often ventilator circuits will also include filters both for incoming air and for exhaled gas. Sometimes, filters are included both where the inhalation tubing and exhalation tubing are attached to the ventilator, with an additional filter attached to the Y-component for increased safety. Inspiratory filters help to prevent patient infection from a potentially infected ventilator, while expiratory filters help to protect the device and the surrounding healthcare staff from contamination from the patient. Sometimes, inspiratory filters are combined with the HME and placed proximate to the Y-piece.

Single limb ventilators are an alternative to dual limb ventilators and do not include distinct inhalation and exhalation pathways. Instead, single limb set ups utilize exhaust valves to externally remove exhaled air, rather than a distinct exhalation pathway back to the ventilator.

Systems for compressing air to generate a positive pressure for ventilators include bellows generators, which use a driving gas to pressurize a bellows to compress the air, and piston ventilators, which utilize an electronic motor to drive compression of a piston within a chamber to compress the gas. Generally, piston ventilators are preferred, given the more precise control over tidal volumes and the potential harmful leakage of the driving gas used in bellows ventilators (especially descending bellows ventilators), among other reasons. One alternative to the pressure solutions of both bellows and piston ventilators is a turbine-based ventilator, which is able to take in air through a turbine to deliver high amounts of that air to support ventilator operations.

Ventilators typically operate using control of volume, pressure, or both, but each of these modes often require precise monitoring from medical professionals to ensure the safety of the patient. A ventilator using pressure control (i.e., configured to deliver a set amount of pressure) is often advantageous, as it allows the device to be operated without risk of barotrauma or other injuries caused by overinflation of the lungs. However, pressure control does not account for an obstruction to inhaling the air from the ventilator, causing the amount of delivered pressure to be insufficient to flow of needed air does not flow into the lungs, resulting in possible morbidity without careful supervision. On the other hand, ventilators utilizing volume control typically are advantageous as they allow operators to set amounts of volume based on monitoring the patient's condition, helping to more precisely reach a desired value of PaCO2. However, left unattended, volume control fails to account for an inability to exhale properly the incoming air, resulting in a buildup of air in the lungs and overpressure, leading to barotrauma or even bursting of the lungs.

Once the control variable (either volume or pressure) is set, then the operating mode is able to be set. The two primary operating modes are assist/control (A/C) and synchronous intermittent mandatory ventilation (SIMV). A/C typically delivers a preset number of mandatory breaths, but allows the patient to also trigger their own breaths. Even if the patient triggers their own breaths, A/C allows the ventilator to entirely deliver the breaths. A/C is good in ensuring that patients utilize less effort to deliver each breath, as the machine does the work, but carries the risk of triggering hyperventilation, which is sometimes lethal. SIMV also allows delivers a preset number of mandatory breaths, but also allows the patient to initiate spontaneous breaths between mandatory breaths. SIMV is able to be run with either pressure control or volume control. SIMV allows patients to contribute more to the breathing, as they are able to initiate the spontaneous breaths and is therefore useful for weaning a patient off support. SIMV helps to maintain respiratory muscles, distributes even tidal breaths, and decreases mean airway pressure. However, other spontaneous modes (relying on spontaneous breaths from patients) are also used, including continuous positive airway pressure (CPAP), pressure support ventilation (PSV), and volume support (VS). Some additional, secondary modes include and/or control mode ventilation (CMV), airway pressure release ventilation (APRV), mandatory minute ventilation (MMV), inverse ratio ventilation (IRV), pressure-regulated volume control (PRVC), proportional assist ventilation (PAV), adaptive support ventilation (ASV), adaptive pressure control (APC), volume-assisted pressure support (VAPS), neurally adjusted ventilatory assist (NAVA), automatic tube compensation (ATC), and/or high frequency oscillatory ventilation (HFOV).

While the range of these modes and the ability of some ventilators to select from different modes is helpful for allowing doctors or other medical professionals to adapt very particularized long-term care to patients based on patient specific conditions, the difficulty of operating ventilator devices keeps them from being able to be operated by an average, non-licensed medical professional. For example, the input variables for ventilators typically at least include tidal volume (V_{T}), positive end-expiratory pressure (PEEP), respiratory rate (RR), and inspiratory airflow (V′). However, the average individual is incapable of understanding how to measure these variables or what they signify. Therefore, a non-trained individual attempting to operate a prior art ventilator is at high risk of injuring the patient being ventilated, perhaps even severely.

Therefore, there is a need for a ventilator that is able to operate sophisticated modes suitable for medically trained personnel, yet enables a user without medical training to operate when respiratory relief is needed, even in emergency situations apart from medical centers. Such a device is preferably able to serve as a complementary device to an automated external defibrillator (AED) in allowing average people to use complex, dangerous medical equipment in emergency situations with low risks of harm.

The present disclosure provides a ventilator suitable for emergency use, such as cardiac pulmonary resuscitation as well as ventilation in non-cardiac induced medical events. The device operates in a mode that allows sophisticated performance with optionally multiple inputs that are used to automatically control the ventilator operation by an onboard controller. The ventilator is able to operate in various modes that are more customary to medically trained personnel, or a combination thereof.

The present system is particularly useful in an emergency situation, particularly within the first eight minutes of a respiratory event, before paramedics are able to arrive. The system functions to provide oxygen to the patient during this time, helping prevent hypoxia, and helps remove CO2 from the patient's body, preventing respiratory acidosis that causes tissue or organ damage. In this way, the system is particularly useful as an at-home unit, a travel appliance, a commercial unit, and/or a disaster response tool, as all of these situations require quick and nimble deployment in the field, where hospital-level resources are not available. Additionally, because the present system utilizes simplified pictorial instructions, it is capable of being operated by a large number of people, literate in a wide variety of languages.

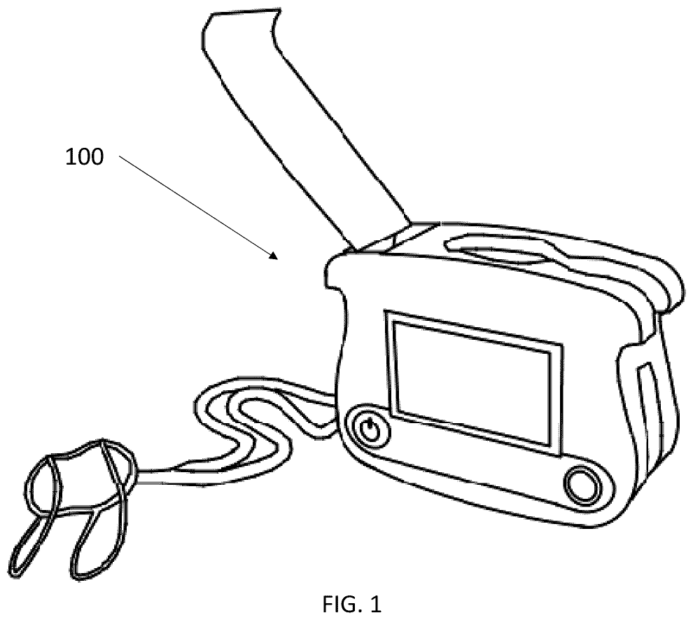

FIG. 1 is an illustrative example of an embodiment of the ventilator that is able to be used, for example, in conjunction with an AED device. The ventilator 100 generally departs from a hospital ventilator in terms of both size and complexity. The ventilator 100 has the capability to perform at a sophisticated level, comparable to that of a hospital ventilator, but avoids complexities in operation in at least some embodiments by a user-friendly interface that requires limited input in order to operate on a less sophisticated level. Based on several simple, easy-to-understand inputs, a non-medical user with minimal training is able to use the ventilator 100, especially in cases of respiratory emergencies. In at least one embodiment, the respirator device is in a form suitable for use with an automated external defibrillator (“AED”) device commonly used for cardiac pulmonary resuscitation (“CPR”). In another embodiment, the respirator is combined with an AED as a single device. In one embodiment, the ventilator 100 is mounted in a cabinet adjacent the AED cabinet for easy access as needed.

Generally, a two-person CPR procedure is recommended having one person to push on a patient's chest to manually pump the patient's heart, while another person breathes air into the patient's lungs to help ventilate the patient for an interim period. The ventilator 100 substitutes for the person respirating the patient, allowing one person to perform the CPR without also having to manually ventilate. In this situation, the ventilator is used in a resuscitator capacity. In one use case example, a patient needs additional air support in their lungs due to a sudden injury, disease, or a chronic condition, which the ventilator 100 is able to provide.

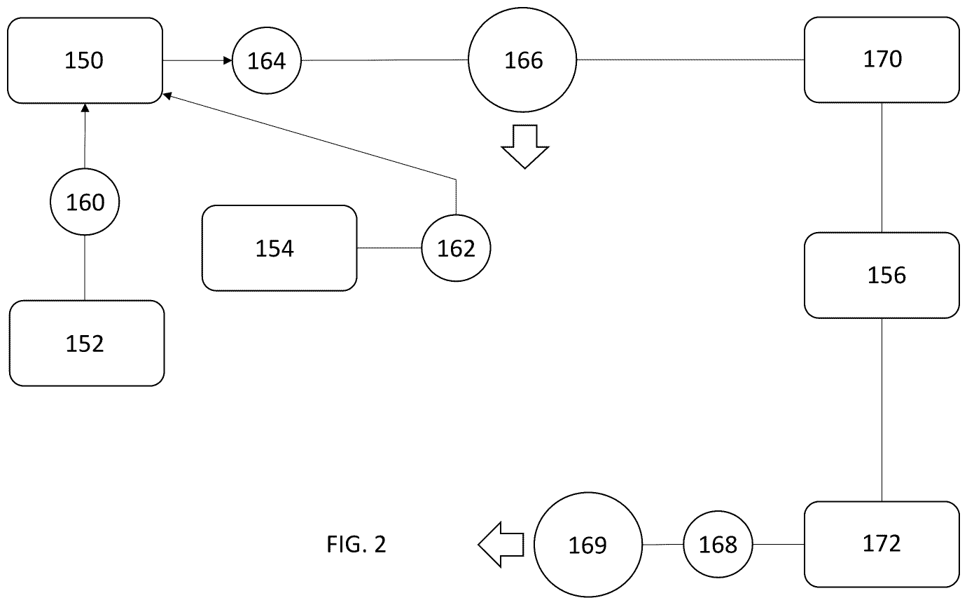

FIG. 2 is a schematic diagram of components of an embodiment of the ventilator system. The ventilator system includes a series of components with a controller that manages the several inputs and provide outputs to the components in various modes. In one embodiment, the ventilator includes, by way of example and not limitation, a ventilator 150 connected to an air supply 152 and/or a supplemental oxygen supply 154, such that the ventilator 150 is able to mix air and supplemental oxygen to deliver to a patient. In one embodiment, the air supply 152 is connected to the ventilator 150 via at least one valve 160 (e.g., a solenoid valve). In one embodiment, the at least one valve 160 includes an ambient air valve, able to control an amount of air that is able to enter a suction port of the ventilator 150. In one embodiment, the system includes at least one air quality sensor operable to detect an air quality entering the ventilator. In one embodiment, the supplemental oxygen supply 154 is connected to the ventilator via at least one valve 162 (e.g., a solenoid valve). Air from the ventilator 150 is transported through tubing to a patient 156. In one embodiment, air from the ventilator 150 passes through at least one valve 164 (e.g., a solenoid valve) before reaching the patient, allowing the at least one valve 164 to regulate the ventilator output. One of ordinary skill in the art will understand that while the supplemental oxygen supply 154 is able to be included in the present invention, it is not necessary in order to operate and the system is capable of using air alone. In one embodiment, the system includes at least one oxygen concentrator in order to increase the percentage of oxygen in the delivered air without providing a supplemental oxygen canister. In one embodiment, the ventilator system includes one or more ampoules of an oxygenating compound. In one embodiment, if the ventilator system detects low oxygen in the inspiratory line, then a preset quantity of the oxygenating compounds is automatically added to the inspiratory line such that they are able to be delivered to the patient.

One of ordinary skill in the art will understand that the ventilator system is not limited to only single limb or dual limb embodiments. In one embodiment, for a single limb design, exhaled gas is released through an exhalation valve. In another embodiment, for a dual limb design, exhaled gas is returned through an exhalation circuit through a first valve 168 (e.g., a solenoid valve). In one embodiment, the exhalation circuit includes at least one positive end-expiratory pressure (PEEP) valve 169, which allows the controller to maintain a pressure in the exhalation pathway greater than atmospheric pressure. In one embodiment, the inhalation line of the circuit includes at least one pressure relief valve 166, able to release an amount of air from the line to reduce the inspiratory pressure of the system, if needed. In one embodiment, the ventilation circuit includes at least one heat and moisture exchanger (HME) for managing temperature and/or humidity of the incoming air. In one embodiment, the inspiratory limb of the system includes at least one heated wire disposed within the tubing that is able to be resistively heated, providing consistent temperature of air to the patient. However, one of ordinary skill in the art will understand that the system is capable of operating without any HMEs or heated wires, as the present invention is intended, most importantly, for short times where temperature and humidity are less critical in comparison to long-term care.

In one embodiment, the system includes at least one inspiratory pressure sensor 170 operable to detect the pressure of the air along the inspiratory pathway of the ventilation circuit and/or at least one exhalation pressure sensor 172 positioned along an exhalation pathway of the ventilation circuit. Data from the at least one inspiratory pressure sensor 170 and/or the at least one exhalation pressure sensor 172 is transmitted to a controller of the ventilation system, and is able to be used to provide feedback for the tidal volume or pressure needed to be delivered by the ventilator 150. In one embodiment, the at least one inspiratory pressure sensor 170 and/or the at least one exhalation pressure sensor 172 are replaced or supplemented with flow rate sensors, operable to detect a flow rate of air entering the patient 156 or gas being exhaled from the patient 156. In one embodiment, flow rate sensors and/or pressure sensors, especially on the inspiratory side, are able to be used to detect when a patient is inhaling or attempting to inhale and send feedback to the controller to modulate the output of the ventilator 150 or the flow allowed by the at least one valve 164 in order to aid the breathing process of the patient 156. In one embodiment, based on parameters of the patient (e.g., height, sex, and/or weight), the system automatically sets a maximum pressure, a minimum pressure, a maximum flow rate, and/or a minimum flow rate for operation. If the pressure sensors or flow rate sensors detect that the system is operating outside of those parameters, the system is operable to automatically change operation of the ventilator (e.g., increase pressure, increase flow rate, decrease pressure, etc.).

In one embodiment, the ventilation includes at least one oxygen sensor and/or at least one carbon dioxide (CO2) sensor. In one embodiment, the at least one oxygen sensor is attached to the inspiratory limb of the ventilation circuit and is able to generate and transmit and O2 concentration of the inspiratory air, such that the controller is able to, for example, control the relative contribution of at least one supplemental oxygen supply to the inspiratory air supply. In one embodiment, the ventilation system is able to be fitted with an external oxygen supply system. This is useful in the event that the ventilator is used in a hospital setting (e.g., after a patient is brought in from using it in the field), as it allows the ventilator to operate with additional equipment to deliver care to the patient. In one embodiment, the at least one CO2 sensor is attached to an expiratory limb of the ventilation system. In one embodiment, the at least one CO2 sensor detects the presence, but not necessarily the concentration, of CO2 in the exhalation pathway to ensure that the patient is actually being assisted in breathing, rather than simply pumping air into the patient that then passes out.

One of ordinary skill in the art will understand that filters (such as bacterial and viral filters) are able to be positioned along various points of the ventilation circuit. By way of example and not limitation, filters are able to be replaced at a position in the ventilation circuit where air leaves the ventilator 150 and/or at a position proximate to where air is delivered to the patient 156 (e.g., connected to a Y-piece). Filters along the inspiratory pathway help to prevent contaminating the patient in the event that the ventilator itself is contaminated. In one embodiment, at least one filter is placed at a position along an exhalation path within the ventilation circuit, which helps to prevent contamination of the ventilator 150 in the event that the patient 156 is sick. In another embodiment, the system does not include any filters.

One of ordinary skill in the art will understand that the mechanism of the ventilator according to the present invention is not intended to be limiting. In a preferred embodiment, the system includes a turbine-based ventilator system. However, in other embodiments, the system includes a piston ventilator, a bellows ventilator, and/or any other type of ventilator.

A display device coupled to the controller accepts input parameters, requests information and displays results and/or a status of the patient. As an example of a typical form of control in hospital respirator devices, a controller includes a first control method correspond to a volume control that controls a volume of inspiratory air delivered to a patient, but does not directly control pressure. Another typical control method is a pressure control method that controls the pressure of inspiratory air, but does not directly control volume.

In one embodiment, most of the elements of the system, including the ventilator, the controller, and the display, are contained within a casing, as shown with the device in FIG. 1. An exception is the tubing for connecting to the patient, as this tubing needs to extend some length and there cannot always be contained within the casing. However, in one embodiment, when the device is not in use, the tubing is able to be collapsed and fit within an internal recess of the casing. In one embodiment, the casing also includes at least one power source (e.g., at least one battery, at least one solar cell, etc.) for powering operations of the ventilator even when not connected to an outlet. In one embodiment, the casing includes at least one port for connecting to an electrical outlet. One of ordinary skill in the art will understand that the type of power port used for the ventilator is able to vary and nothing in the present invention is intended to be limiting in that regard.

In one embodiment, the casing includes at least one geolocation sensor (e.g., at least one Global Positioning System (GPS) chip). In one embodiment, the at least one geolocation sensor is integrated with at least one cellular card. In one embodiment, the casing includes at least one wireless module operable to communicate data from the ventilator system over a network (e.g., via cellular connection, via a wireless local area network (WLAN), via a wireless personal area network (WPAN), via satellite communications, etc.) to a central server and/or to one or more specific recipients. In one embodiment, when the ventilation system is activated, the casing automatically transmits a signal to a nearby hospital or medical office including the geolocation data, such that medical professionals are able to locate and assist the patient in distress. In one embodiment, the ventilation system is able to pair with at least one external user device (e.g., a cellular phone, a laptop, a tablet, etc.) and automatically transmits the geolocation and/or other sensor data from the casing to the external user device. The external user device is then able to use a software application to select at least one nearby medical office or hospital and automatically transmits data from the ventilation system to the medical office or hospital, allowing the medical professionals to be informed about a real time status and location of the patient. In one embodiment, the ventilation system includes at least one speaker operable to play at least one emergency beacon sound. By playing this loud noise, medical professionals are able to locate the patient after arriving in the geolocated position, which is especially useful if the area is crowded.

In one embodiment, the controller automatically determines initial operating parameters for the ventilation system based on simple patient characteristics input through an associated input device (e.g., an attached display, a cell phone, a computer, a tablet, etc.). In one embodiment, the initial operating parameters include tidal volume, respiratory rate, and/or pressure. In one embodiment, the respiratory rate is preset between approximately 10 and 12 breaths per minute. In one embodiment, in order to determine the initial operating parameters, the controller utilizes at least one table having corresponding values for tidal volume, respiratory rate, and/or pressure for each input height and/or for each combination of height with other characteristics (e.g., sex, weight, etc.). In one embodiment, the initial operating parameters include maximum pressure, minimum pressure, maximum air flow, and/or minimum air flow for the sensors. In one embodiment, the controller is operable to automatically change at least some of the operating parameters (e.g., tidal volume, pressure, etc.) based on readings of inspiratory or expiratory pressure or flow rate sensors detecting pressures or flow rates outside of the tolerable range for the particular patient. In this way, the system is able to be responsive to a patient's current condition, rather than overpressurize or under supply the patient's lungs. In another embodiment, the controller includes at least one artificial intelligence module operable to automatically determine the initial operating parameters and/or to change the operating parameters during use.

In one embodiment, upon activation of the ventilation system, the ventilation system automatically attempts to connect to at least one network (e.g., a cellular network, a WLAN network, a WPAN network, a satellite network, etc.) and automatically retrieves and provides updated data from a cloud serving, adjusting operation of the ventilation system. By way of example and not limitation, if new research indicates it is optimal to provide mandatory breaths at respiration rates between 8-9 breaths per minute, rather than 10-12 breaths per minute, then the ventilation system is updated to automatically provide 8-9 breaths per minute (when in a mode having mandatory breaths). Additionally, if new patient data indicates different optimal tidal volumes or pressures for patients of a given height, then the tables utilized by the controller are able to be automatically updated to reflect this new data.

In one embodiment, the device begins operating in a spontaneous operation mode, such that it detects attempted breaths based on negative pressure readings and then is able to supplement those breaths. In this embodiment, the device does not begin by providing mandatory breaths, but only works as a supplemental tool. In one embodiment, the spontaneous operation mode includes pressure support ventilation (PSV). In one embodiment, if the device does not detect any spontaneous breaths for a preset amount of time (e.g., 10 seconds), then the device automatically switches to providing mandatory breaths in a new control mode. In one embodiment, the new control mode includes an assist control (A/C) or synchronized intermittent mandatory ventilation (SIMV) mode, but one of ordinary skill in the art will understand that the system is not limited to those modes. In one embodiment, if the system detects a preset number of spontaneous breaths and/or a preset threshold frequency of spontaneous breaths, then the system automatically switches back to a spontaneous operation mode. One of ordinary skill in the art will understand, however, that the present system is not limited to beginning in a spontaneous mode and, in another embodiment, begins in a non-spontaneous control mode where a set number of mandatory breaths are delivered. The ability of the system to respond to differences in flow rate and pressure allows the system to act entirely autonomously, without the need for an untrained user to manually adjust the settings over time.

The user interface provides a user-friendly experience to an untrained or minimally trained user. For the purposes of this application, minimally trained will be understood to encompass those individuals having basic life support (BLS) training and/or advanced cardiovascular life support (ACLS) training, but not having further formal medical education. The respirator device includes, as referenced above, pictogram instructions, simplified terminology in common parlance, universal signs, color, sound, lights that activate for different steps or functions, voice instructions, and other methods of communicating procedures, requests for information, status, and results. Such features and methods of interaction allow use in various environments including homes, schools, and businesses for untrained or minimally trained users, as well as use by more highly trained medical personnel.

As an example of operational input, the ventilator prompts for a patient's height. A database, such as a look-up table or a formula for direct calculation or other correlation of input to ventilator settings, is used to determine appropriate volume at some pressure for a starting point in inspiration. As the ventilator operates, sensor feedback adjusts the volume and/or pressure as needed for an end result that is beneficial with the system operating in an automatic operation. As discussed above, if the system has choices of simulated modes, then the relevant parameter(s) are adjusted only, unless for example, other parameters fall outside of a safety zone to cause the system to override the settings of the selected parameters.

For more accuracy, the ventilator requests one or more additional characteristics of the patient, including weight, sex, any known current condition, such as a current cardiac arrest and other current status, and even past known relevant medical history. The patient inputs are correlated to ventilator parameters for at least initial start of operation.

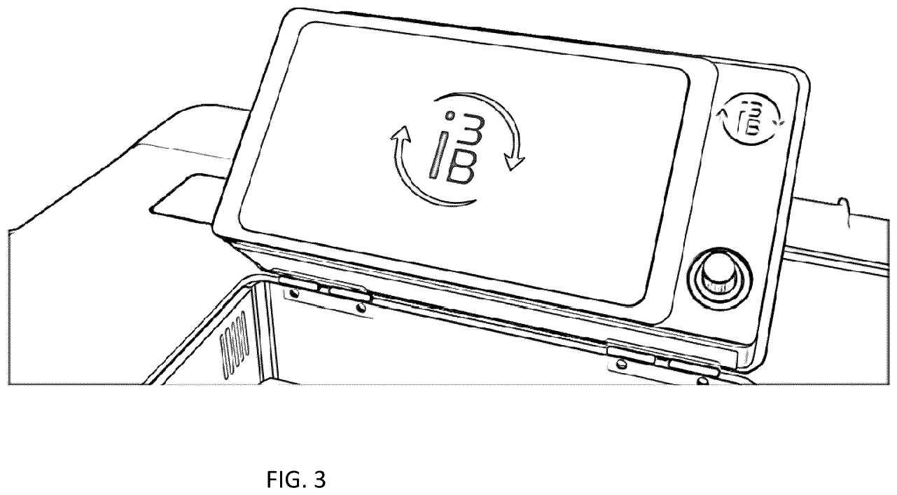

FIGS. 3-9 are exemplary user interface screens in at least one embodiment for simplified operation by untrained or minimally trained users that allow the exemplary ventilator to function in a sophisticated manner. One of ordinary skill in the art will understand that the user interface screens shown are only illustrative and are able to be selectively offered, modified, or not offered in various embodiments, and that the inputs shown entered into each screen are able to be made optional in some embodiments of the present invention.

FIG. 3 is an illustrative startup screen. The activity on the screen, with FIG. 3 showing rotation of arrows around a central logo, indicates to the user the system is operational and starting up. In one embodiment, the startup screen includes an indication of whether the system is able to access a network, including whether an emergency signal was successfully transmitted to at least one medical professional. In this way, if the startup screen indicates that such a signal failed to go through, then the operator knows that alternate methods need to be used to contact medical professionals.

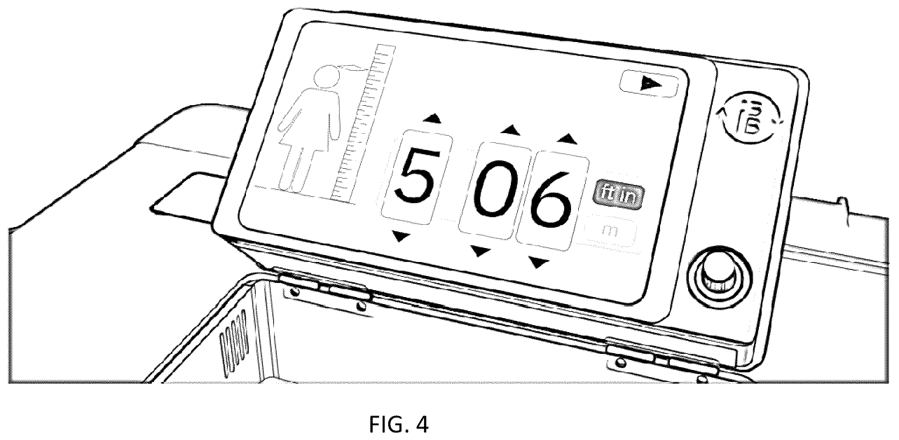

FIG. 4 is an illustrative user interface screen for height input. In one embodiment, the respirator device operates on the basis of a patient's height using statistical data for selecting estimated respiratory parameters for at least an initial regimen. An advantage of using height as a parameter is that it is easily estimated even by a user with little medical knowledge or other knowledge of the patient, even compared to other visual characteristics, such as weight. In one embodiment, as shown in FIG. 4, height is selected by selecting arrow key buttons that increment or decrement values up and down by one. In one embodiment, the screen is able to receive a selection to change units between metric and imperial units (or other length unit systems), allowing users from different backgrounds to use the system. In one embodiment, height is selected with a slider interface, with a shortest height (or zero) on the left and a tallest height (say, 10 feet) on the right. By moving the slider between these values, the user is able to select a height that matches the patient. In one embodiment, as the slider moves, a pictorial graphic of a user on the screen changes in height, providing more visual feedback for operation of the slider. In one embodiment, the casing of the device includes at least one measuring tape operable to extend out of the casing such that the patient is able to be measured in real time if the operator feels uncertain about their intuitive measurement.

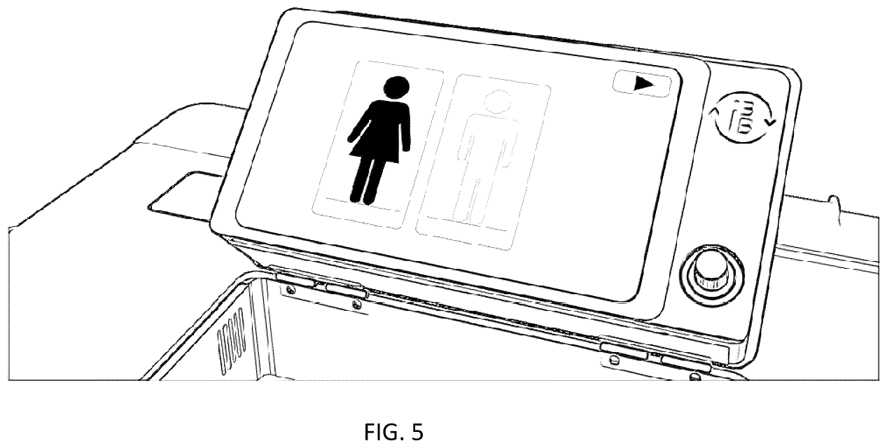

FIG. 5 is an illustrative user interface screen for sex input. In lieu of or in addition to height, the patient sex provides some operating parameters for at least an initial setting of the respirator device that the controller is then able to tune, if appropriate, based on results from sensor readings and particularly the patient's inspiratory and expiratory readings as applicable.

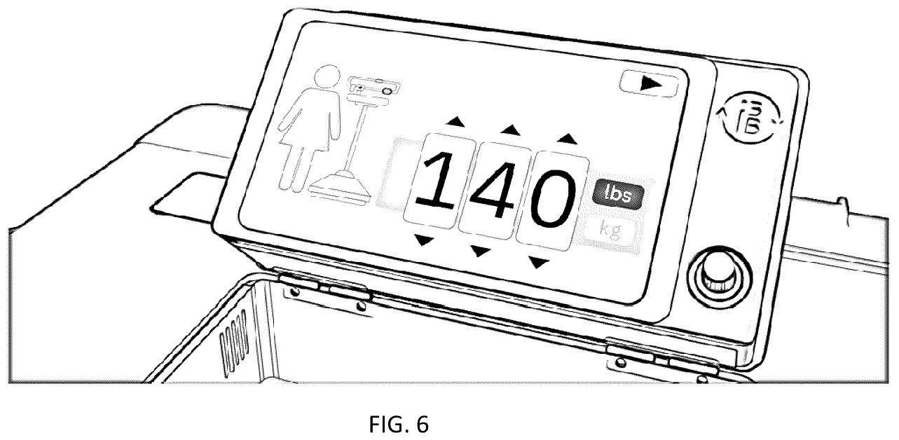

FIG. 6 is an illustrative weight input interface. The weight is estimated or actually determined if the patient is conscious and knows their weight or is able to stand on a weight scale. One of ordinary skill in the art will understand that weight is not necessary to include if both height and sex are input, but that in conjunction with the height and/or sex selections, weight sometimes helps to refine the operating parameters. In one embodiment, the weight input interface is able to receive a selection to switch between metric and imperial units of measurement. In one embodiment, weight is selected with a slider interface, with a smallest weight (or zero) on the left and a largest weight (say, 1000 lbs., 50 kg, etc.) on the right. By moving the slider between these values, the user is able to select a weight that matches the patient. In one embodiment, as the slider moves, a pictorial graphic of a user on the screen changes in size, providing more visual feedback for operation of the slider.

In one embodiment, the system further includes an age input interface. The age is estimated or actually determined if the patient is conscious and knows their age or has an ID that indicates their date of birth. One of ordinary skill in the art will understand that age is not necessary to include in the event that both height and sex are input, but that in conjunction with the height and/or sex selections, age sometimes helps to refine the operating parameters. In one embodiment, age is selected with a slider interface, with a youngest age (or zero) on the left and a largest age (say, 115 years) on the right. By moving the slider between these values, the user is able to select an age that matches the patient. In one embodiment, as the slider moves, a pictorial graphic of a user on the screen appears to age, providing more visual feedback for operation of the slider. In one embodiment, if an age is selected that falls outside of the acceptable range of use for the device (e.g., the patient is too young for the system), then the system automatically provides a warning and will not initiate care, but is still able to contact medical authorities. In one embodiment, if the device is unable to be used on a particular patient, the device automatically displays pictorial instructions for how to ventilate the individual without the use of the device.

In one embodiment, the device does not include a confirmation screen wherein the user needs to confirm that quantities such as tidal volume, respiratory rate, etc. for the system are correct based on the input patient attributes (e.g., height, weight, gender, etc.). This is helpful, as it does not put the burden of understanding complex medical terminology on a potentially unsophisticated user or potentially confuse the user and prevent the device from being activated as quickly as is necessary to save a patient.

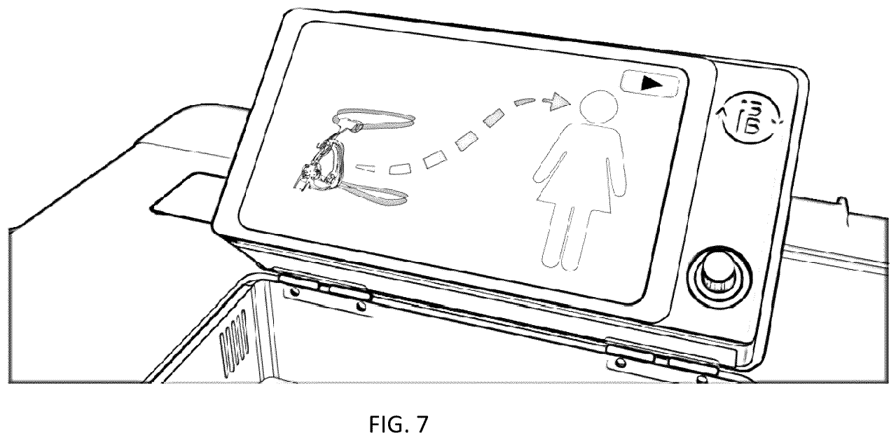

FIG. 7 is an illustrative instructions screen. The instructions are preferably pictographic and/or animated to provide clarity to even untrained or minimally trained users. While not expressly shown, it is understood that other screens are possible including output screens on the patient, alarm indicators, and other instructions if follow up actions are needed such as a mask needing adjustment to avoid a leak and so forth. In one embodiment, if the ventilator system receives input that the patient is also undergoing cardiac arrest, at least one instructions screen is generated with a pictorial graphic indicating where on the patient's chest to perform compressions. In one embodiment, the ventilation system provides an indication of timing (e.g., a periodically flashing color, a metronome beat, etc.) for performing the compressions.

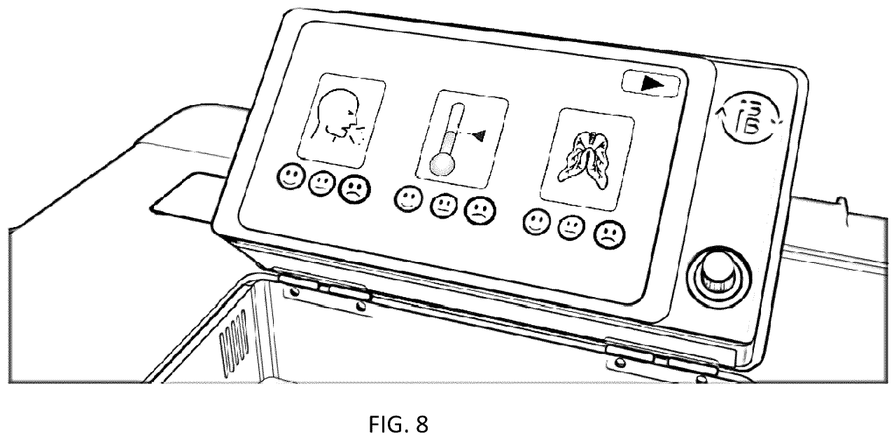

FIG. 8 is an illustrative user interface screen for conditions of the patient. In one embodiment, the conditions include, for example, how well the patient is breathing, patient temperature, or other conditions (e.g., whether the patient is also having a heart attack). In one embodiment, each condition is symbolized by a pictorial graphic image in addition to or in lieu of text, allowing for easier use by operators who are literate in different languages.

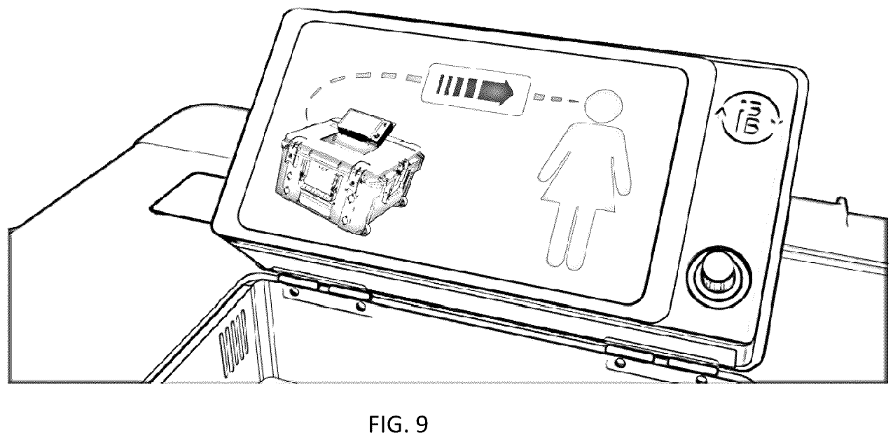

FIG. 9 is an illustrative user interface screen for commencing operation. With the initial input, the ventilator commences operations of the respiration. The pictographic instructions assist in directing user actions. In one embodiment, voice instructions in an appropriate language are used.

In one embodiment, the ventilator system is operable to receive voice commands to provide the input characteristics for the patient (e.g., height, weight, sex, age, etc.). In one embodiment, the ventilator system is operable to prompt each step through auditory messages.

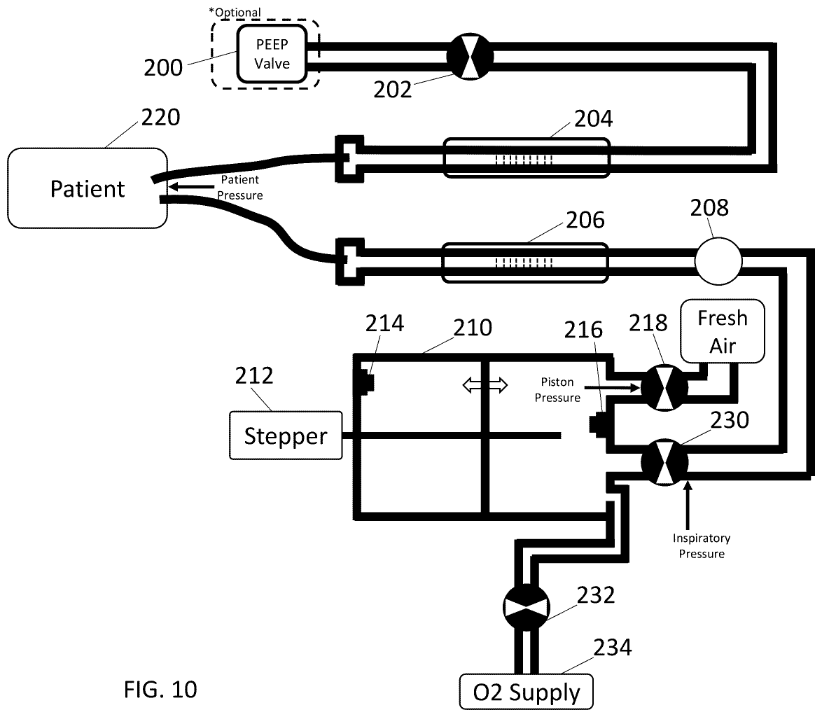

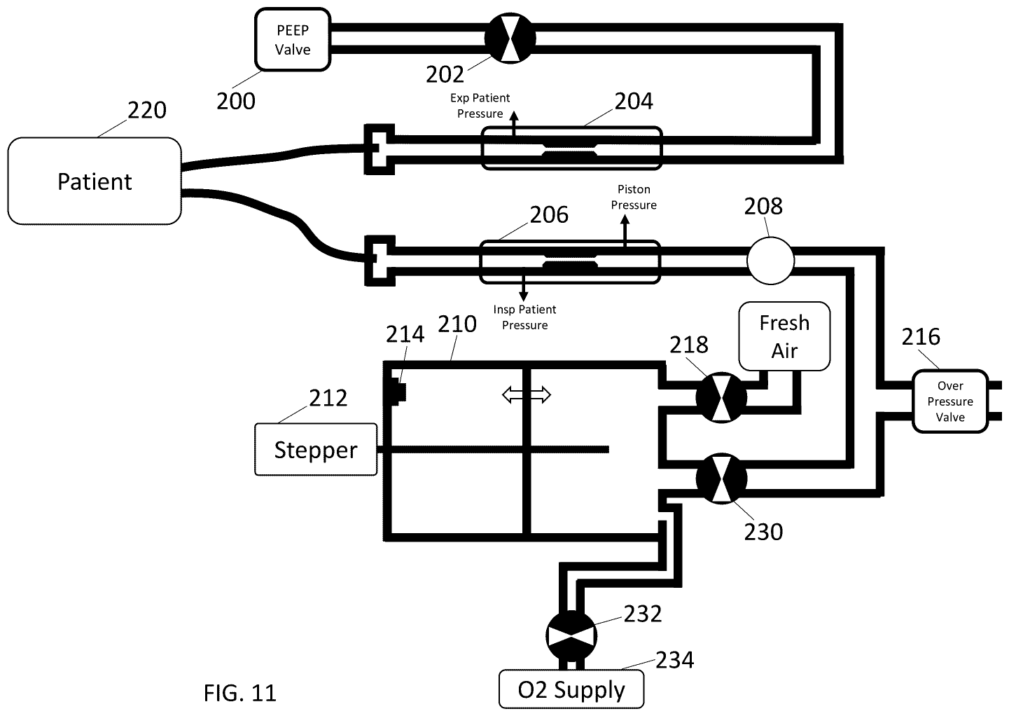

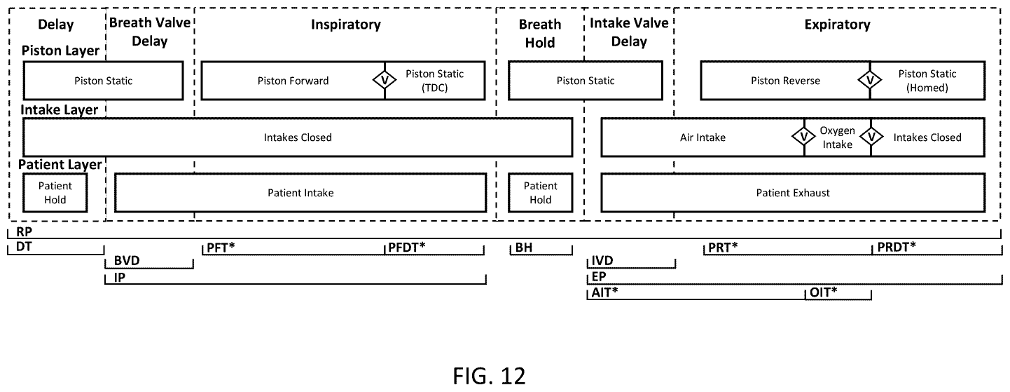

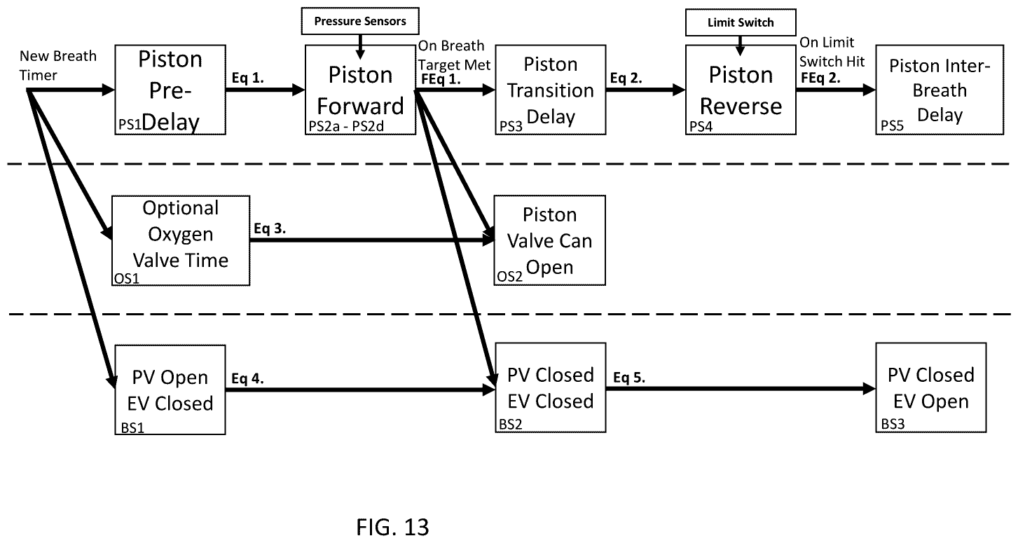

FIG. 10 is a schematic diagram of inspiratory and exhalatory circuits for a ventilator according to one embodiment of the present invention. FIGS. 10 and 11 show examples of ventilation circuits able to be used with the present invention. One of ordinary skill in the art will understand that the specific orientation of the device or components of the device shown in FIGS. 10 and 11 are not intended to be limiting, but rather provide one embodiment for how the invention is able to function. In particular, although a piston-based system is shown in FIGS. 10 and 11 and discussed in relation to FIGS. 12 and 13, the present invention is also compatible for use with non-piston based ventilation systems (e.g., with turbine or bellows-based systems).

In one embodiment, a patient 220 is connected to two tubes, allowing for separate exhalation and inhalation pathways, as is common for many ventilator designs. The exhalation pathway 204 includes one or more expiratory valves 202, which are able to be opened to allow carbon dioxide-rich exhaled air to exit the patient, while preventing backflow of air from the surrounding environment. In one embodiment, the exhalation pathway 204 is able to include one or more PEEP valves 200 to ensure sufficient expiratory pressure exists such that the alveoli of the lungs of the patient do not collapse. The exhalation pathway is able to include one or more sensors operable to produce data gathered by the microcontroller of the ventilator to inform any changes to operation and/or to update a patient ventilation profile on a connected micro computer for adjusting hyperparameters of the system. In one embodiment, the one or more sensors include one or more expiratory flow sensors, one or more expiratory pressure sensors, one or more expiratory O2 sensors, one or more expiratory CO2 sensors, and/or one or more other types of sensors.

The inhalation pathway 206 includes an inspiratory flow generator 210, in this case a piston-based system (but also inclusive at least of a turbine-based system). The piston-based system includes a stepper motor 212 in communication with a microcontroller, such that the microcontroller is operable to activate or deactivate the piston, or to adjust parameters of the motor-driven actuation of the piston. In turbine-inclusive embodiments, the microcontroller is operable to adjust parameters of the turbine to achieve a similar effect. In one embodiment, the inspiratory flow generator 210 includes at least one home safety switch 214, operable to cut power supply to the ventilator in the event of power surges or other events to prevent potential sparks or shocks. In one embodiment, the inspiratory flow generator 210 includes at least one limit switch 216, operable to turn off the device, or send alerts to adjust parameters of the device in the event that the inspiratory flow generator 210 acts outside of safe parameters (e.g., pressure within the inspiratory flow generator 210 is too high, O2 concentration is too high or two low, etc.).

The inspiratory flow generator 210 is connected to at least one air source (e.g., the surrounding environment or a more isolated source of air). At least one air intake valve 218 is included and operable to limit the supply of air to the inspiratory flow generator 210 from the at least one air source as needed. Additionally, the inspiratory flow generator 210 is connected to at least one O2 supply 234. At least one oxygen intake valve 232 is included in a line connecting the inspiratory flow generator 210 and the O2 supply 234, and is operable to limit O2 supplied to the inspiratory flow generator 210. The relative amounts of air and oxygen supplied to the inspiratory flow generator 210 are able to be adjusted as needed with control of the corresponding valves to ensure that a proper O2 concentration is present in the inspiratory flow, while the inspiratory flow generator 210 produces sufficient pressure so as to allow the patient 220 to breathe.

The inhalation pathway 206 further includes at least one inspiratory valve 230, configured to limit supply of the inspiratory air flow to the patient 220 as appropriate and to prevent backflow of exhaled air from the patient 220 into the inspiratory flow generator 210. In one embodiment, the inhalation pathway 206 includes at least one oxygen sensor 208. In one embodiment, the inhalation pathway 206 includes one or more additional sensors able to produce data to be supplied to a microcontroller, such that parameters of the system are able to be adjusted based on the sensor data. In one embodiment, the one or more additional sensors include one or more inspiratory flow sensors, one or more inspiratory pressure sensors, one or more piston pressure sensors, one or more CO2 sensors, and/or one or more other types of sensors. In one embodiment, as shown in FIG. 11, the inhalation pathway 206 further includes at least one overpressure valve 236 configured to allow release of some of the inspiratory flow in the event that inspiratory pressure is too high. In one embodiment, the inhalation pathway 206 and/or the exhalation pathway 204 include one or more Venturi throats.

The inclusion of O2 and CO2 sensors is particularly beneficial due in the use of the system by non-trained medical professionals. In the event that supraglottal intubation is necessary, detection of abnormal differential levels of O2 and CO2 helps to determine whether the tube is properly intubated or whether it is feeding to a patient's stomach. In one embodiment, the GUI of the system is operable to display an alert and/or instructions in the event that abnormal differential levels of O2 and/or CO2 are detected that indicate a likely error in intubation.

FIG. 12 is a chart illustrating functions of different parts of a ventilation system across different phases of operation and controlling variables for each phase according to one embodiment of the present invention. FIG. 12 outlines different functions performed by different components and conceptual layers of the ventilation system across a breath cycle, with indications of corresponding variables for each portion of the cycle provided below. As mentioned above, while FIG. 12 is particularized to a piston-based system, this figure is only meant to be illustrative of the type of division of functions present in ventilators and is not intended to be limiting as to the structure of the present invention. Furthermore, with regard to the variables described below, the variables that correlate to structural components of the piston system are merely meant to be illustrative of the types of programmable and controllable variables of the present invention, and one of ordinary skill in the art will understand that corresponding variables exist and are able to be controlled for turbine-based systems according to the present invention.

Prior art ventilators have traditionally operated in discrete modes, such as those mentioned above, including pressure support modes, volume support modes, and numerous other more advanced modes of operation. Some prior art ventilators are further capable of operating in multiple modes or even switching modes during functioning, often as a result of detecting spontaneous breaths from a user. However, the idea of distinctive modes having particular variables that are controlled and particular variables that are fixed is limiting, even where the ventilators are capable of switching between these modes. The use of these discrete modes, each of which are often useful in particular cases, but potentially damaging in other cases, are useful when operated by trained professionals who are capable of identifying when to use particular modes. However, the use of distinctive modes presents a challenge for using the ventilator in emergency situations by non-trained medical personnel, as such a person is unlikely to know what mode to select and, if they chose wrong, could potentially do more harm than good for the patient. Furthermore, using discrete modes conceptually limits the ventilator from acting in “in-between” spaces, represented by combinations of variables that would not otherwise fit in a particular preset mode, but which still represent valid and even useful ways to ventilate the patient. For example, in volume assist or pressure-regulated volume control (PRVC), tidal volume is preset. In pressure assist and pressure support, pressure is preset. In SIMV operation, either pressure or volume are preset, depending on the specific mode.

The present invention addresses this issue by providing “modeless” operation. In one embodiment, the present invention does not require selection of any mode by an operator, but instead operates as a continuous control system, freely controlling a wide range of variables for ventilation, without any variables necessarily being designated as “fixed” or “controllable.” In this way, the ventilator acts as a “software-defined ventilator,” rather than a hardware defined ventilator. As such, the ventilator both provides for a more user-friendly and safe operation by non-trained professionals and is more responsive to the individual patient. Because the ventilator is able to freely set the whole range of variables, it is possible that, at any given moment, the ventilator approximates the functionality of a particular preset mode, but without the ventilator being constrained to operation within that modality for any amount of time.

A first layer, called the piston layer, represents the physical movement of the piston within the system, namely whether the piston is static, moving forward, or reversing (i.e., moving backward). The second layer, called the intake layer, shows when the piston system receives air and/or oxygen from corresponding gas supplies, and when the intakes from the gas supplies are closed. Finally, the patient layer represents when the patient is inhaling air, exhaling air, or doing neither (and correspondingly when inspiratory and expiratory valves are open or closed). The timing of each phase of the various layers, and the timing of overlap between different phases of multiple layers are not hard-set and represent directly controllable variables, or derived variables influenced from other controllable variables.

Examples of controllable and derived variables are described below. Controllable variables belong to at least three categories: structural, operational, and boundary-based. Structural variables are directly related to specific timings of physical components of the system (e.g., piston speed) that are able to be directly influenced. Operational variables represent target parameters of the system, which are achieved through manipulation of different physical settings of the system, but which are not themselves direct physical changes to the system (e.g., target flow rate, respiratory rate, etc.). Finally, boundary-based variables represent maximum and/or minimum conditions of operation that the system ensures that it does not exceed so as to maintain, for example, the health of the patient.

Structural variables include, but are not limited to, delay time (DT), breath valve delay (BVD), breath hold (BH), intake valve delay (IVD), and/or piston return speed. As represented in FIG. 12, DT represents the interval of time during between when the patient has finished exhaling and when the patient begins inhaling, when the intakes of the system are closed, and the piston remains static. This period is able to be varied, changing the sensitivity of the system to spontaneous breaths by the patient. BVD represents the interval time of between when the patient begins inhaling and when the piston begins moving. BH represents the interval time between when a patient is finished inhaling and when the patient exhales. BH is able to be influenced by when valves open and close and when expiratory pressure is applied. IVD represents the period of time between when a patient begins expiring and when the piston begins reversing. Finally, piston return speed represents the rate at which the piston retracts.

Operational variables include, but are not limited to, fraction of inspired oxygen (FiO2), respiratory rate (RR), expiratory/inspiratory ratio (EIR), target tidal volume, target peak pressure, and target flow rate. RR represents the rate at which the patient breathes, able to be represented in breaths per second or breaths per minute, which is often important to change depending on the state of the user (e.g., different for sleeping vs. waking, etc.). FiO2 represents a percentage of oxygen in the air delivered to the patient. Target tidal volume, pressure, and flow rate represent ideal parameters that the system attempts to achieve.

Boundary-based variables include, but are not limited to, minimum and maximum tidal volumes, minimum and maximum peak inspiratory pressures, and minimum and maximum flow rates. Rather than representing specific parameters to achieve for the system, each of these represent maximum and minimum values, where the system is able to adjust operations if variables are found to be outside of or approaching the boundaries of these ranges.

The controllable parameters are able to be used to calculate derived variables that are also important for functioning. For example, the respiratory period (RP) is equal to the reciprocal of the respiratory rate. The active time (AT) of the system is equal to the respiratory rate minus the delay time and the breath hold (i.e., only when the patient is actively inhaling or exhaling). The inspiratory period (IP) is the active time multiplied by 1 minus the EIR, while the expiratory period (EP) is equal to the active time multiplied by the EIR.

The piston forward time (PFT*) is calculated according to Equation 1 below, while the piston forward dead time (PDFT*) is equal to IP−BVD−PFT.

$$PFT = \frac{T {V}_{target}}{{FLOW}_{target} * 1000} * ( IP - BVD ) \qquad ( Equation 1 )$$

The piston position (PPo) is able to be determined by integration based on timing, and the piston return time (PRT) is equal to the PPo multiplied by 1000 divided by the piston return speed. The piston return dead time (PRDT) is equal to EP−IVD−PRT. The oxygen intake time (OIT) is measured as a function of FiO2 and the piston return time. The air intake time (AIT) is equal to PRT−OIT+IVD.

FIG. 13 is a chart illustrating ventilator state machine logic according to one embodiment of the present invention. The operation of each layer of the ventilator is further shown in FIG. 13, with the actions of the piston shown at the top, the action of the intake system shown in the middle, and actions of the patient inspiratory valve (PV) and expiratory valve (EV) shown at the bottom. After a delay period, the piston moves forward and stops when the breath target is met. After a brief delay, the piston reverses, after which another delay period begins. For the intake system, the oxygen valve opens first followed by the air valve (i.e., the piston valve can), although the oxygen valve opening is able to be skipped depending on the FiO2 data and depending on the situation. Finally, the patient inspiratory and expiratory valves alternatingly open and close depending on the part of the breathing cycle.

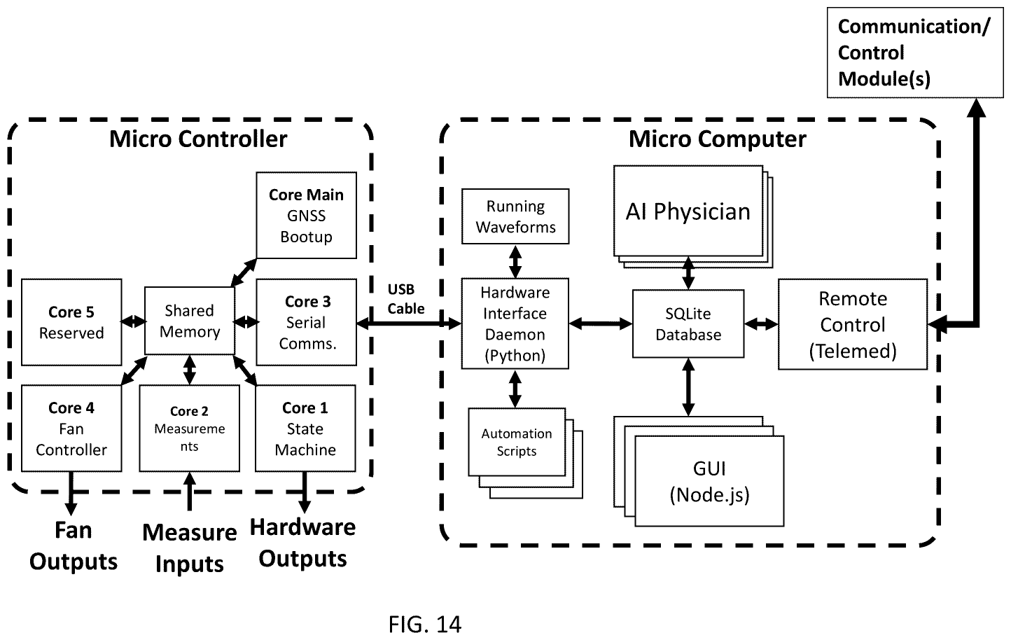

FIG. 14 is a schematic diagram illustrating communication between a micro controller and a microcomputer for a ventilator according to one embodiment of the present invention. An additional advantage of the present invention over prior art ventilators is the ability of the system to construct a digital twin of the patient based on the sensor data and any user input of parameters (e.g., height, weight, sex, age, etc.), and then utilize this data to update models for how optimally to ventilate the patient. The system in particular is able to use artificial intelligence (AI) to update the model, including one or more controllable parameters for the ventilator based on the digital twin. However, the system is able to do this without relying on the AI or any network connection for the basic functioning of the ventilator, as the system is also able to act deterministically.

The system accomplishes this combination via a division of the functionality of the microcontroller and microcomputer, and further by the use of separate, independent cores of the microcontroller (e.g., with independent cycle times). For example, one core of the microprocessor is able to be used for the state machine for directing hardware outputs. Another core is able to be used specifically for handling sensor measurements or user input measurements and passing this information to the state machine. Another core is able to be specifically dedicated to communication with the microcomputer for coordinated the AI-based updating of the model. Another core is able to be used for controlling a fan of the ventilator, while yet another core is able to be reserved for other functionality. The main core of the microcontroller is able to be used for bootup and for geolocation services. Each of these cores is able to share and access the same memory of the microcontroller. Independence of these cores allows, for example, the state machine to continue functioning even if the communication unit fails for any reason.

The communication unit of the microcontroller communicates with a hardware interface daemon of the microcomputer to provide sensor and user input data from the microcontroller for allowing the microcomputer to construct a digital twin of the patient. The microcomputer is then able to create waveforms based on that data as part of the digital twin and utilize an artificial intelligence unit to generate parameter changes to send to the microcontroller to update the model implemented by the state machine. The microcomputer includes a database (e.g., a SQLITE database) for storing information and is operable to include a GUI for displaying information regarding the parameters. The microcomputer is able to include a remote control operable to communicate with a remote communication control module.

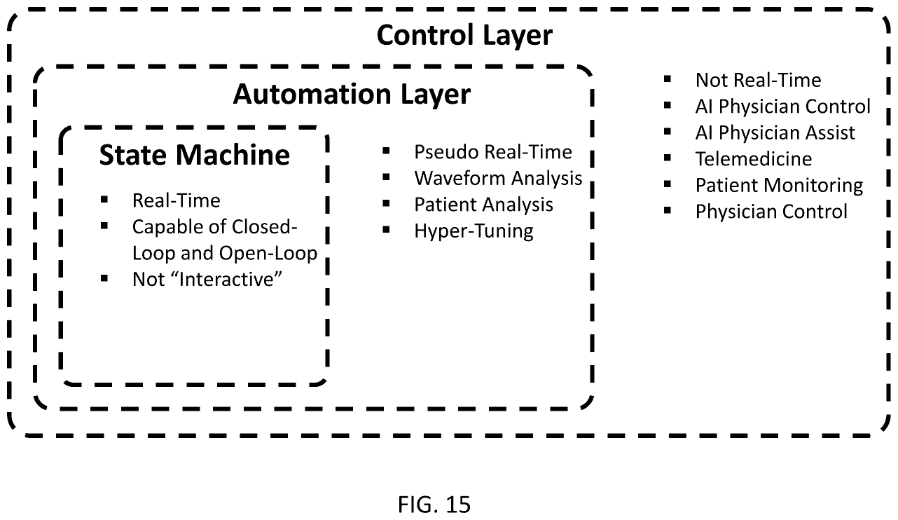

FIG. 15 is a schematic diagram showing different functions of different logical layers of a ventilation system according to one embodiment of the present invention. The system of the present invention essentially includes three layers of control, allowing for the system to be robust in immediately responding to a patient, while also allowing the model to adjust and improve over time. The state machine level, performed at the microcontroller, is able to operate and respond in real time and is capable of both utilized closed-loop and open-loop operations. The state machine itself is not interactive, although parameters for the state machine are able to be updated based on determinations of the automation layer. The automation layer is represented by the microcomputer and operates in pseudo real-time, with minimal delay for communication with the microcontroller and for processing the data. The automation layer constructs digital twins of the patient for waveform analysis and for hyper-tuning of parameters of the model of the state machine to update the model. Furthermore, an additional control layer, in communication with the automation layer, is able to be used, which allows for larger AI models or for direct, manual physician manipulation of the parameters of the state machine, or of the AI models in the automation layer. At the control layer, new research regarding optimizing ventilator parameters is able to be incorporated in order to update the state machine or the AI models of the automation layer. Furthermore, the control layer is able to be incorporated as part of a telemedicine system for providing patient data to a physician or provider.

In a modeless system, key ventilatory parameters are continuously monitored and dynamically adjusted. Each parameter directly influences how the ventilator delivers breaths. For example, when the system sets a relatively low respiratory rate (RR) and an increased inspiratory pressure while controlling flow rate, the ventilator is able to mimic a pressure control mode, without rigidly controlling only traditional pressure control-related variables. This pseudomode allows the system to deliver pressure-targeted breaths to allow for the lung compliance of the patient and to shape the delivered volume. On the other hand, if RR is set high with a fixed tidal volume, the ventilator is able to operate similarly to a volume control mode, delivering predictable tidal volumes at set intervals. The system is able to seamlessly transition between these states, not having a set mode, by simply adjusting the outcome-focused parameters (e.g., RR, tidal volume, inspiratory pressure, etc.).

Other adjustable parameters, such as FiO2, do not necessarily help to simulate any existing mode, but continuous control of FiO2 does allow for a highly responsive automated oxygenation strategy. In one embodiment, pulse oximetry data and/or arterial blood gas measurements are integrated with sensor data and control of the ventilator to allow for seamless shifting of FiO2 parameters in real time, regardless of how the other parameters are set.

In one embodiment, by lengthening the inspiratory phase and limiting expiration, the ventilator of the present invention is able to replicate modes that favor alveolar recruitment and improve oxygenation (e.g., pressure control modes for Acute Respiratory Distress Syndrome (ARDS) patients). Alternatively, in one embodiment, by shortening inspiratory phases with longer expiratory phases, the ventilator is able to better accommodate patients with obstructive lung disease, simulating modes with prolonged expiration (e.g., similar to custom settings in pressure control or volume control for Chronic obstructive pulmonary disease (COPD) patients).

In one embodiment, by gradually decreasing tidal volume while maintaining a given RR to reduce lung injury, the ventilator is able to provide a “lung protective” strategy without formally switching to a lung protective mode. Additionally, when the ventilator automatically adjusts flow and pressure to achieve a volume target, the system simulates a volume control logic. Alternatively, by fixing target pressure limits and allowing volume to vary, the system is able to simulate a pressure-control mode.

In one embodiment, by adjusting trigger sensitivity and flow delivery dynamically, the ventilator is able to seamlessly shift from a full mandatory breath delivery pattern (e.g., analogous to a controlled ventilation mode) to a more support pattern that assists the efforts of the patient (e.g., analogous to pressure support or CPAP modes). In one embodiment, by decreasing mandatory RR and increasing trigger sensitivity, the ventilator is able to transition to a pattern analogous to a spontaneous breathing mode. In one embodiment, when a patient initiates a breath, the ventilator automatically responds by increasing flow as needed, emulating a pressure-support ventilation mode, without formally switching to a pressure support mode.

In one embodiment, by incrementally increasing PEEP, the ventilator shifts from a low support setting to one maintaining alveolar recruitments, similar to ARDS ventilation strategies. In one embodiment, by lowering peak inspiratory pressure after a period of high pressure support, the ventilator is able to automatically, gradually weaning the patient off high-pressure ventilation, transitioning towards spontaneous breathing conditions, similar to moving from a controlled mode or a supported (or even CPAP) mode, all via continuous parameter adjustment, rather than discrete mode switching.

By operating outside a “mode-based approach” in line with the exemplary embodiments provided above, the system of the present invention instead embodies a modular, “composable” architecture. Each operational capability and each individually controllable variable are able to be introduced or refined independently without forcing the entire system into a rigid mode change. This provides for a flexible system, where new features are layered on top of existing functionality without disrupting established controls. As a result, the ventilator is able to quickly adapt to the changing physiology of a patient, blending multiple strategies and providing safer, more stable respiratory support, especially in emergency situations. The control algorithms are more akin to a closed loop system that continuously adapts to patient data and user inputs, rather than forcing an operator to choose a listed named mode from a menu. Due to the lack of strict mode boundaries, transitions between strategies are able to be much smoother, thereby reducing risk to a victim. This also reduces the risk of operator error, especially by non-trained personnel using the device. Furthermore, for both non-medical personnel and clinical staff, the modeless operation provides for easier training and support, as it does not require an operator to memorize the effects and benefits of various modes, instead allowing for greater automation and for focus on the actual parameters that need to be adjusted to support a patient.

In one example, a patient is gradually recovering and beginning to take more spontaneous breaths. With a mode based embodiment, the clinician intends to increase the autonomy of the patient by moving from a fully controlled mode to a partially supportive mode, but accidentally selects a mandatory ventilation mode that does not support spontaneous breaths. This potentially causes, at beast, discomfort, and, at worst, a potentially fatal respiratory crisis. However, if using the modeless operation of the present invention, the operator of the ventilator (or an AI module) is able to adjust parameters such as trigger sensitivity and mandatory breath frequency, while increasing pressure support. The control algorithms are then able to be used to smoothly transition toward a spontaneous breathing pattern.

In a second example, a patient with ARDS requires a lung protective strategy with low tidal volumes, moderate to high PEEP, and careful pressure control. In a mode-based system, a volume control mode with a preset tidal volume that is too large for the injured lungs of the patient is able to be accidentally selected, leading to inflammatory damage and decreased changes of recovery. In the modeless system of the present invention, however, tidal volume is able to be reduced, inspiratory pressure limited, and PEEP increased to shift the device toward a protective strategy, without the risk of an incorrect mode being selected.

In one embodiment, the software included on the ventilator includes built-in reference tables, standard formulas, or advanced algorithms for determining safe, effective operating parameters (e.g., target tidal volumes, airway pressure limits, and flow rates). This allows for entry of simpler, and more readily understood, parameters (e.g., height, age, etc.), causing the ventilator to automatically calculate an initial ideal tidal volume and respiratory rate, at minimum.

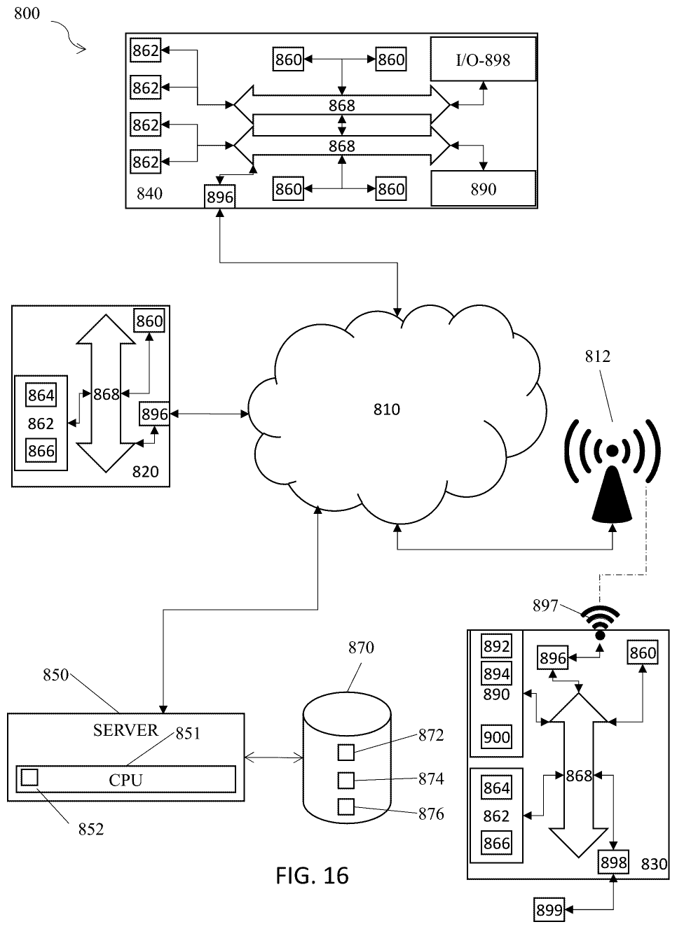

FIG. 16 is a schematic diagram of an embodiment of the invention illustrating a computer system, generally described as 800, having a network 810, a plurality of computing devices 820, 830, 840, a server 850, and a database 870.

The server 850 is constructed, configured, and coupled to enable communication over a network 810 with a plurality of computing devices 820, 830, 840. The server 850 includes a processing unit 851 with an operating system 852. The operating system 852 enables the server 850 to communicate through network 810 with the remote, distributed user devices. Database 870 is operable to house an operating system 872, memory 874, and programs 876.

In one embodiment of the invention, the system 800 includes a network 810 for distributed communication via a wireless communication antenna 812 and processing by at least one mobile communication computing device 830. Alternatively, wireless and wired communication and connectivity between devices and components described herein include wireless network communication such as WI-FI, WORLDWIDE INTEROPERABILITY FOR MICROWAVE ACCESS (WIMAX), Radio Frequency (RF) communication including RF identification (RFID), NEAR FIELD COMMUNICATION (NFC), BLUETOOTH including BLUETOOTH LOW ENERGY (BLE), ZIGBEE, Infrared (IR) communication, cellular communication, satellite communication, Universal Serial Bus (USB), Ethernet communications, communication via fiber-optic cables, coaxial cables, twisted pair cables, and/or any other type of wireless or wired communication. In another embodiment of the invention, the system 800 is a virtualized computing system capable of executing any or all aspects of software and/or application components presented herein on the computing devices 820, 830, 840. In certain aspects, the computer system 800 is operable to be implemented using hardware or a combination of software and hardware, either in a dedicated computing device, or integrated into another entity, or distributed across multiple entities or computing devices.

By way of example, and not limitation, the computing devices 820, 830, 840 are intended to represent various forms of electronic devices including at least a processor and a memory, such as a server, blade server, mainframe, mobile phone, personal digital assistant (PDA), smartphone, desktop computer, netbook computer, tablet computer, workstation, laptop, and other similar computing devices. The components shown here, their connections and relationships, and their functions, are meant to be exemplary only, and are not meant to limit implementations of the invention described and/or claimed in the present application.

In one embodiment, the computing device 820 includes components such as a processor 860, a system memory 862 having a random access memory (RAM) 864 and a read-only memory (ROM) 866, and a system bus 868 that couples the memory 862 to the processor 860. In another embodiment, the computing device 830 is operable to additionally include components such as a storage device 890 for storing the operating system 892 and one or more application programs 894, a network interface unit 896, and/or an input/output controller 898. Each of the components is operable to be coupled to each other through at least one bus 868. The input/output controller 898 is operable to receive and process input from, or provide output to, a number of other devices 899, including, but not limited to, alphanumeric input devices, mice, electronic styluses, display units, touch screens, gaming controllers, joy sticks, touch pads, signal generation devices (e.g., speakers), augmented reality/virtual reality (AR/VR) devices (e.g., AR/VR headsets), or printers.

By way of example, and not limitation, the processor 860 is operable to be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that perform calculations, process instructions for execution, and/or other manipulations of information.

In another implementation, shown as 840 in FIG. 16, multiple processors 860 and/or multiple buses 868 are operable to be used, as appropriate, along with multiple memories 862 of multiple types (e.g., a combination of a DSP and a microprocessor, a plurality of microprocessors, one or more microprocessors in conjunction with a DSP core).

Also, multiple computing devices are operable to be connected, with each device providing portions of the necessary operations (e.g., a server bank, a group of blade servers, or a multi-processor system). Alternatively, some steps or methods are operable to be performed by circuitry that is specific to a given function.

According to various embodiments, the computer system 800 is operable to operate in a networked environment using logical connections to local and/or remote computing devices 820, 830, 840 through a network 810. A computing device 830 is operable to connect to a network 810 through a network interface unit 896 connected to a bus 868. Computing devices are operable to communicate communication media through wired networks, direct-wired connections or wirelessly, such as acoustic, RF, or infrared, through an antenna 897 in communication with the network antenna 812 and the network interface unit 896, which are operable to include digital signal processing circuitry when necessary. The network interface unit 896 is operable to provide for communications under various modes or protocols.

In one or more exemplary aspects, the instructions are operable to be implemented in hardware, software, firmware, or any combinations thereof. A computer readable medium is operable to provide volatile or non-volatile storage for one or more sets of instructions, such as operating systems, data structures, program modules, applications, or other data embodying any one or more of the methodologies or functions described herein. The computer readable medium is operable to include the memory 862, the processor 860, and/or the storage media 890 and is operable be a single medium or multiple media (e.g., a centralized or distributed computer system) that store the one or more sets of instructions 900. Non-transitory computer readable media includes all computer readable media, with the sole exception being a transitory, propagating signal per se. The instructions 900 are further operable to be transmitted or received over the network 810 via the network interface unit 896 as communication media, which is operable to include a modulated data signal such as a carrier wave or other transport mechanism and includes any delivery media. The term “modulated data signal” means a signal that has one or more of its characteristics changed or set in a manner as to encode information in the signal.

Storage devices 890 and memory 862 include, but are not limited to, volatile and non-volatile media such as cache, RAM, ROM, EPROM, EEPROM, FLASH memory, or other solid state memory technology; discs (e.g., digital versatile discs (DVD), HD-DVD, BLU-RAY, compact disc (CD), or CD-ROM) or other optical storage; magnetic cassettes, magnetic tape, magnetic disk storage, floppy disks, or other magnetic storage devices; or any other medium that stores the computer readable instructions and which are accessible by the computer system 800.

In one embodiment, the computer system 800 is within a cloud-based network. In one embodiment, the server 850 is a designated physical server for distributed computing devices 820, 830, and 840. In one embodiment, the server 850 is a cloud-based server platform. In one embodiment, the cloud-based server platform hosts serverless functions for distributed computing devices 820, 830, and 840.

In another embodiment, the computer system 800 is within an edge computing network. The server 850 is an edge server, and the database 870 is an edge database. The edge server 850 and the edge database 870 are part of an edge computing platform. In one embodiment, the edge server 850 and the edge database 870 are designated to distributed computing devices 820, 830, and 840. In one embodiment, the edge server 850 and the edge database 870 are not designated for distributed computing devices 820, 830, and 840. The distributed computing devices 820, 830, and 840 connect to an edge server in the edge computing network based on proximity, availability, latency, bandwidth, and/or other factors.

It is also contemplated that the computer system 800 is operable to not include all of the components shown in FIG. 16, is operable to include other components that are not explicitly shown in FIG. 16, or is operable to utilize an architecture completely different than that shown in FIG. 16. The various illustrative logical blocks, modules, elements, circuits, and algorithms described in connection with the embodiments disclosed herein are operable to be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans are able to implement the described functionality in varying ways for each particular application (e.g., arranged in a different order or partitioned in a different way), but such implementation decisions should not be interpreted as causing a departure from the scope of the present invention.

It must be understood that the inventions disclosed and taught herein are susceptible to numerous and various modifications and alternative forms. The use of a singular term, such as, but not limited to, “a,” is not intended as limiting of the number of items. Further, the various methods and embodiments of the system are able to be included in combination with each other to produce variations of the disclosed methods and embodiments. Discussion of singular elements are also inclusive of plural elements and vice-versa. References to at least one item include one or more items. Also, various aspects of the embodiments are used in conjunction with each other to accomplish the understood goals of the disclosure. Unless the context requires otherwise, the term “comprise” or variations such as “comprises” or “comprising,” should be understood to imply the inclusion of at least the stated element or step or group of elements or steps or equivalents thereof, and not the exclusion of a greater numerical quantity or any other element or step or group of elements or steps or equivalents thereof. The term “coupled,” “coupling,” “coupler,” and like terms are used broadly herein and include any method or device for securing, binding, bonding, fastening, attaching, joining, inserting therein, forming thereon or therein, communicating, or otherwise associating, for example, mechanically, magnetically, electrically, chemically, operably, directly or indirectly with intermediate elements, one or more pieces of members together and are able to further include without limitation integrally forming one functional member with another in a unity fashion. The coupling is able to occur in any direction, including rotationally. The device or system is able to be used in a number of directions and orientations. The order that steps are able to occur in a variety of sequences unless otherwise specifically limited. The various steps described herein are combined with other steps, interlineated with the stated steps, and/or split into multiple steps. Some elements are nominated by a device name for simplicity and would be understood to include a system or a section, such as a controller encompasses a processor and a system of related components that are known to those with ordinary skill in the art and may not be specifically described. Various examples are provided in the description and figures that perform various functions and are non-limiting in shape, size, description, but serve as illustrative structures that are able to be varied as would be known to one with ordinary skill in the art given the teachings contained herein. As used herein, “patient” refers to a person in need of respirator assistance and is not restricted to a person under the care of a physician or medical center.

The above-mentioned examples are provided to serve the purpose of clarifying the aspects of the invention, and it will be apparent to one skilled in the art that they do not serve to limit the scope of the invention. By nature, this invention is highly adjustable, customizable and adaptable. The above-mentioned examples are just some of the many configurations that the mentioned components are able to take on. All modifications and improvements have been deleted herein for the sake of conciseness and readability but are properly within the scope of the present invention.

The invention claimed is:

1. A portable emergency ventilator system, comprising:
an inspiratory tubing and an expiratory tubing configured to connect to a patient;
an inhalation flow generator connected to the inspiratory tubing;
a plurality of sensors connected with the inspiratory tubing, the expiratory tubing, and/or the inhalation flow generator;
a microcontroller including a plurality of independent cores; and
a microcomputer in communication with the microcontroller;
wherein the microcontroller sends sensor data generated by the plurality of sensors to the microcomputer;
wherein an artificial intelligence (AI) module of the microcomputer generates a digital twin of the patient with waveforms constructed from the sensor data; and
wherein the AI module generates updates to models for operating the ventilator system and wherein the microcomputer sends messages including the updates to the models to the microcontroller.

2. The ventilator system of claim 1, wherein one of the plurality of independent cores of the microcontroller includes a state machine with operating parameters for the ventilator system.

3. The ventilator system of claim 1, wherein the microcomputer is further operable to communicate with at least one remote control module.

4. The ventilator system of claim 1, wherein one of the plurality of independent cores of the microcontroller includes a communication module configured to manage the communication with the microcomputer.

5. The ventilator system of claim 1, wherein the plurality of sensors includes at least one flow rate sensor, at least one pressure sensor, at least one tidal volume sensor, at least oxygen sensor, and/or at least one carbon dioxide sensor.

6. The ventilator system of claim 1, wherein the AI module generates the updates to the models for operating the ventilator system based on the digital twin of the patient and input receiving regarding the patient from a graphical user interface (GUI).

7. The ventilator system of claim 1, wherein the ventilator system is not set to operate in a particular mode.

8. The ventilator system of claim 1, wherein the inhalation flow generator includes a turbine-based system or a piston-based system.

9. The ventilator system of claim 1, wherein the plurality of independent cores of the microcontroller are connected with and able to read a shared memory of the microcontroller.

10. A portable emergency ventilator system, comprising:
a plurality of sensors configured to generate sensor data regarding pressure, flow rate, tidal volume, oxygen content, and/or other information regarding the ventilator system;
a microcontroller including a plurality of independent cores; and
a microcomputer in communication with the microcontroller;
wherein one of the plurality of independent cores of the microcontroller includes a state machine configured to control operating parameters of the ventilator system;
wherein the microcontroller sends sensor data generated by the plurality of sensors to the microcomputer;
wherein an artificial intelligence (AI) module of the microcomputer generates a digital twin of the patient with waveforms constructed from the sensor data;
wherein the AI module generates updates to models for operating the ventilator system and wherein the microcomputer sends messages including the updates to the models to the microcontroller; and
wherein the state machine of the microcontroller automatically updates the parameters of the ventilator system based on the messages from the microcomputer.

11. The ventilator system of claim 10, wherein the microcomputer is further operable to communicate with at least one remote control module.

12. The ventilator system of claim 10, wherein one of the plurality of independent cores of the microcontroller includes a communication module configured to manage the communication with the microcomputer.

13. The ventilator system of claim 10, wherein the plurality of sensors includes at least one flow rate sensor, at least one pressure sensor, at least one tidal volume sensor, at least oxygen sensor, and/or at least one carbon dioxide sensor.

14. The ventilator system of claim 10, wherein the AI module generates the updates to the models for operating the ventilator system based on the digital twin of the patient and input receiving regarding the patient from a graphical user interface (GUI).

15. The ventilator system of claim 10, wherein the ventilator system is not set to operate in a particular mode.

16. The ventilator system of claim 10, wherein the plurality of independent cores of the microcontroller are connected with and able to read a shared memory of the microcontroller.

17. A method for operating a ventilator system, comprising:
connecting an inspiratory tubing and an expiratory tubing to a patient;
operating an inhalation flow generator to provide air to the patient through the inspiratory tubing;
a microcontroller of the ventilator system sending sensor data from a plurality of sensors connected to the inspiratory tubing, the expiratory tubing, and/or the inhalation flow generator to a microcomputer;
an artificial intelligence (AI) module of the microcomputer generating a digital twin of the patient with waveforms constructed from the sensor data; and
the AI module generating updates to models for operating the ventilator system and sending the updates to the microcontroller.

18. The method of claim 17, further comprising the microcomputer communicating with at least one remote control module.

19. The method of claim 17, wherein one of the plurality of independent cores of the microcontroller includes a communication module configured to manage the communication with the microcomputer.

20. The method of claim 17, further comprising the AI module generating the updates to the models for operating the ventilator system based on the digital twin of the patient and input receiving regarding the patient from a graphical user interface (GUI).
